(19) United States
(12) Patent Application Publication
(10) Pub. No.: US 2003/0163181 A1
(43) Pub. Date: Aug. 28, 2003

(54) Protection of neurological tissue by direct CNS perfusion cooling

(75) Inventors: Glenn D. Frazer, Wynnewood, PA (US); David P. Hesson, Malvern, PA (US); Timothy J. Pelura, Malvern, PA (US); James C. Barnitz, Schwenksville, PA (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Neuron Therapeutics, Inc., Malvern, PA (US)

(21) Appl. No.: 10/314,870

(22) Filed: Dec. 9, 2002

Related U.S. Application Data
(60) Provisional application No. 60/337,978, filed on Dec. 7, 2001.
(60) Provisional application No. 60/339,859, filed on Dec. 12, 2001.

(51) Int. Cl. A61F 7/00

(52) U.S. Cl. 607/105

(57) ABSTRACT

Provided, among other things, is a method of treating, in an animal, an injury that is stroke, traumatic brain injury including traumatic surgical brain injury, brain hemorrhage other than stroke, spinal cord traumatic injury including spinal cord surgical trauma, infection, ischemia caused by surgical intervention, CNS injury caused by cardiac arrest, encephalitis or encephalomyelitis not due to infection, comprising: (a) creating a flow pathway, with an instillation catheter and an effluent catheter, that provides liquid flow in the vicinity of the injury; (b) instilling into the flow pathway an cerebrospinal perfusion fluid having a temperature from (i) 4° C. as a lower temperature to (ii) 4° C. below normal temperature of the animal; and (c) maintaining perfusion through the flow pathway for at least 12 hours of the cerebrospinal perfusion fluid having a temperature, at the point of instillation, from (i) 4° C. as a lower temperature to (ii) 4° C. below normal temperature of the animal.

This application claims priority of U.S. Ser. No. 60/339,978, filed Dec. 7, 2001 and U.S. Ser. No. 60/339,859, filed Dec. 12, 2001.

This invention relates to a method of producing directed cooling of neural tissue (hypothermia) which is useful for treating neurologic diseases and conditions that cause death or damage to neurological tissue, such as stroke, traumatic brain injury (TBI), brain hemorrhage, spinal cord traumatic injury, infection, ischemia caused by surgical intervention or ischemic injury caused by cardiac arrest.

It has been understood for 50 years that lowering body temperature to 28-35° C. provides protection of organs and tissues (Bigelos et al., 1950). Hypothermia is most often induced via external cooling of the entire body by application of ice, cold fluids, cooling blankets or all of these techniques to the skin of the patient. These methods provide total body (global) hypothermia to the patient effecting all organ systems. This approach has significant drawbacks in the treatment of neurologic diseases or conditions. First, the time required to cool the entire body is long and the length of time the entire body can be cooled is limited (Ringelstein et al., Neurology 42:289-98, 1992). This is very important in an emergency situation. Second, global cooling causes cardiovascular problems, such as cardiac arrhythmias, reduced cardiac output, increased systemic vascular resistance, pulmonary edema, increased blood viscosity, coagulopathy and leukopenia. These effects may result in damage to the heart and other organs, (McCallum, Canadian J. Surgery 6:457-60, 1990).

Heat exchange devices have been described by Dobak (U.S. Pat. No. 6,149,677) or Noda (U.S. Pat. No. 6,146,411) that are placed within blood vessels to cool specific organs or the entire body by cooling blood flowing to an organ. This method has the draw backs that when applied to a large or diffusely located organ the device is small and lacks the capacity to cool the organ to useful hypothermic temperatures of 20-30° C., or if adapted to apply at larger scale to large blood vessels would run the risk of cooling the entire body with the same problems mentioned above.

Frazer et al (Neurology 41 (Suppl 1):806S, March 1991) described perfusion into the lateral ventricles of the brain and out the cisterna magna to induce brain hypothermia in cats at fluid flow rates of 1-5 mL/min. The perfusate was an oxygen-carrying emulsion.

Despite the problems with global hypothermia there are indications that it can protect neural tissue damage. In animal models of stroke (cerebral ischemia), induction of global hypothermia via externally applied cooling was shown to reduce infarct size up to 90%, (Baker et al., J. Neurosurg. 77:438-44, 1992; Zhang et al., Stroke 25:522-3, 1993). In human clinical studies global hypothermia induced by externally applied cooling seems to also be effective in the treatment of acute stroke, (Schwab et al., Stroke 29:2461-6, 1998; Keller et al. Neurosurgery Focus, vol. 8(5), 2000; Wijdicks, Mayo Clin. Proc. 75:945-52, 2000; Kammersgaard et al., Stroke 31:2251-6, 2000). Hypothermia was shown to protect ischemic neural tissue during surgical procedures to repair arteries that supplied the brain, (Demaria et al. J. Cardiovasc. Sura. 41:299-302, 2000) and aortic surgery (Takano et al., Eur. J. Cardio-Thoracic Surg. 18:262-9, 2000). In animal models of pneumococcal meningitis, induction of global hypothermia via externally applied cooling was shown to reduce inflammatory response and to inhibit intracranial hypertension, (Angstwurm et al., J. Cerebral Blood Flow and Metabolism 20:834-838, 2000).

The cerebrospinal fluid (CSF) pathway system, which intimately bathes and permeates brain and spinal cord tissues, constitutes a circulatory system within the body. Although it has some similarities to systemic vascular and lymphatic circulation, its anatomical arrangement differs considerably. Indeed, this system has been named the “third circulation” system. Due to the extensive area of CSF-tissue contact over the cerebral and spinal cord surfaces, in the miniature Virchow-Robins spaces, and cerebral ventricles, the cerebrospinal fluid system constitutes a vast, complex and intimate avenue for access to central nervous tissue.

Disclosed herein are superior methods of treating critical diseases and conditions of neuronal tissue using a cooling (4-33° C.) perfusion (20-80 mL/min) of that tissue, methods of scrubbing out toxic metabolic by-products with appropriate fluids, methods of treating certain neurologic diseases and conditions. The method disclosed allows rapid cooling of the target tissue under very control conditions and controlled warming of the tissue under the same controlled conditions. The agents disclosed are less expensive and easier to prepare than other agents. These methods are not disclosed in such documents as U.S. Pat. No. 4,446,155, which assert without discussion that one can produce hypothermia by perfusion of an oxygenated emulsion.

### SUMMARY OF THE INVENTION

In one embodiment, the invention provides a method of treating, in an animal, an injury that is stroke, traumatic brain injury including traumatic surgical brain injury, brain hemorrhage other than stroke, spinal cord traumatic injury including spinal cord surgical trauma, infection, ischemia caused by surgical intervention, CNS injury caused by cardiac arrest, encephalitis or encephalomyelitis not due to infection, comprising: (a) creating a flow pathway, with an instillation catheter and an effluent catheter, that provides liquid flow in the vicinity of the injury; (b) instilling into the flow pathway an cerebrospinal perfusion fluid having a temperature from (i) 4° C. as a lower temperature to (ii) 4° C. below normal temperature of the animal; and (c) maintaining perfusion through the flow pathway for at least 12 hours of the cerebrospinal perfusion fluid having a temperature, at the point of instillation, from (i) 4° C. as a lower temperature to (ii) 4° C. below normal temperature of the animal.

In another embodiment, the invention provides a method of treating a brain or spinal injury comprising: (1) creating a flow pathway, with an instillation catheter and an effluent catheter, that provides liquid flow in the vicinity of the injury; (2) instilling into the flow pathway a cerebrospinal perfusion fluid having a temperature from (i) 4° C. as a lower temperature to (ii) 4° C. below normal temperature, wherein the cerebrospinal perfusion fluid does not have a respiration-supporting amount of oxygen; and (3) maintaining perfusion of the cerebrospinal perfusion fluid having a temperature from (i) 4° C. as a lower temperature to (ii) 4° C. below normal temperature through the flow pathway for at least 12 hours. The method can, for example, be applied where the injury is one listed above in this section of the specification.

The instillation methods of the invention can include (A) monitoring the temperature of effluent cerebrospinal perfusion fluid from the flow pathway; and (B) providing a signal when temperature of the effluent reaches 15° C., (or 17 or 18° C.) the signal comprising (a) a feedback signal for a controller that decreases the flow rate of instillation or increases the temperature of the instilled cerebrospinal perfusion fluid, or (b) an alarm.

In another embodiment, the invention provides a method of treating in an animal a brain or spinal injury, comprising: (a) creating a flow pathway, with an instillation catheter and an effluent catheter, that provides liquid flow in the vicinity of the injury; (b) instilling into the flow pathway an cerebrospinal perfusion fluid having a temperature from (i) 4° C. as a lower temperature to (ii) 4° C. below normal temperature of the animal; (c) thereafter gradually, for a pre-designated amount of time or pursuant to a pre-designated schedule, (i) elevating the temperature of the instilled cerebrospinal perfusion fluid or (ii) decreasing the flow rate of the instilled cerebrospinal perfusion fluid to elevate the temperature of cerebrospinal perfusion fluid exiting the effluent catheter; and (d) in conjunction with the gradual elevating or decreasing, monitoring intracranial pressure and, should the intracranial pressure indicate undue swelling of neural tissue, doing one or more of (i) maintaining a temperature or flow rate for more than the pre-designated time or delaying the schedule of elevating temperature or decreasing flow, (ii) decreasing the temperature of the instilled cerebrospinal perfusion fluid, or (iii) increasing the flow rate of the instilled cerebrospinal perfusion fluid.

In still another embodiment, the invention provides a method of delivering an cerebrospinal perfusion fluid into the cerebral spinal pathway of an animal, comprising: (1) initiating flow of the cerebrospinal perfusion fluid into a ventricular catheter, through a cerebral spinal pathway, and out a lumbar outflow catheter with the patient in a supine position and using a first flow rate, wherein a positive first value for an outlet pressure is maintained in plumbing from the lumbar outflow catheter; (2) increasing flow to a second flow rate greater than the first in conjunction with decreasing the outlet pressure to a second, negative value; and (3) thereafter maintaining flow of the cerebrospinal perfusion fluid into the ventricular catheter with the temperature of the physiologically acceptable liquid from (i) 4° C. as a lower temperature to (ii) 4° C. below normal temperature of the animal. Preferably, between initiating flow to the first rate and increasing to the second rate, adjusting the flow rate to an intermediate rate between the first and second rate in conjunction with adjusting the outlet pressure to a value between the first and second value. Preferably, at some point after finishing the flow at the first rate and prior to the maintaining, inclining the patient

### BRIEF DESCRIPTION OF THE DRAWINGS

FIGS. 1 and 2 display schematically a flow apparatus for flow through the cerebral spinal pathway.

FIG. 3 shows how the patient can be inclined during administration to achieve high flow rates.

FIG. 4 shows an efflux flow rate control mechanism.

FIG. 5 shows a pressure break device.

FIG. 6 illustrates another device for controlling pressure at the drainage end.

### DETAILED DESCRIPTION OF THE INVENTION

In addition to providing CNS physiologically compatible cooling fluids into cerebrospinal passageways, the inventive method can also be used to remove neurotoxic by-products while optionally providing, glucose, electrolytes and essential amino acids into neural tissue. If used in a rapidly exchanging cerebrospinal fluid perfusion system, such as is described in U.S. patent application Ser. No. 09/454,893, filed Mar. 12, 1999 or U.S. patent application Ser. No. 09/619,414, filed Jul. 19, 2000 (the perfusion systems described in these patent documents are incorporated by reference, see below), the inventive composition and methods can be used both to supply these nutrients and, at the same time, remove metabolic waste.

Fluid for Cerebrospinal Perfusion

In one embodiment, the cerebrospinal perfusion fluid (“CSPF”) is a physiologic CNS nutrient solution according to the following:

| | | More | Still More |
|---|---|---|---|
| | Preferred | Preferred | Preferred Range |
| Component | Range | Range | or Amount |
| Albumin, g/dL, | 0.1-2.5 | 1.0-2.2 | 1.87 |
| α-Ketoglutaric Acid, μg/mL | 5-100 | 22-34 | 28 |
| Amino Acids, μg/mL | | | |
| L-Isoleucine | 0.1-10 | 0.2-5 | 0.6 |
| L-Leucine | 0.5-20 | 1-6 | 1.4 |
| L-Valine | 0.5-15 | 1-6 | 1.9 |
| L-Alanine | 1-20 | 1-6 | 3.2 |
| L-Serine | 1-20 | 1-6 | 2.8 |
| L-Histidine | 0.1-10 | 1-6 | 2.1 |
| L-Methionine | 0.05-5 | 0.1-1 | 0.2 |
| L-Phenylalanine | 0.1-10 | 0.2-5 | 0.9 |
| L-Lysine | 1-20 | 1-6 | 3.0 |
| L-Threonine | 1-20 | 1-6 | 3.3 |
| L-Arginine | 1-20 | 1-6 | 2.1 |
| L-Tyrosine | 0.1-10 | 0.2-5 | 0.9 |
| Na⁺, mM | 135-150 | 137-147 | 147 |
| K⁺, mM | 2.5-3.9 | 2.7-3.5 | 2.9 |
| Cl⁻, mM | 110-135 | 116-135 | 130 |
| Ca⁺², mM | 1.0-1.6 | 1.0-1.5 | 1.15 |
| Mg⁺², mM | 0.8-1.6 | 1.0-1.5 | 1.12 |
| Glucose (dextrose), mg/dL | 40-140 | 50-120 | 94 |

The pH of the perfusion fluid, is in the physiological range, such as about 7.3. In one embodiment, the amino acids include tryptophan. In certain embodiments of the invention, the CSPF can be formulated as an emulsion with a molecule effective to carry oxygen, for example, as in one of the formulations described in PCT/US01/22539.

The fluid for cerebrospinal perfusion is preferably formulated such that it is physiologic and can directly contact tissues of the neuraxis for an extended period of time, from hours to days, without causing side effects. For best performance, it is believed that the artificial cerebrospinal fluid should be appropriately buffered and have appropriate amounts of amino acids, electrolytes and other compounds helpful to healthy metabolism. Thus, in preferred methods, these components do not need to be supplied through equilibration with other body fluids. Of course, simpler solutions, such as appropriately balanced salts, are used in neurosurgery and are to some degree acceptable. Where the fluid for cerebrospinal perfusion is formulated with nutrients, it can be termed “artificial cerebrospinal fluid” or “ACSF.”

In some embodiments, the CSPF is simplified. For example, the nutrient components can be omitted. Ions are maintained to the degree required to avoid damage to cerebrospinal tissue. Appropriate amounts of oncotic agents to achieve fluid flow rates of 20-80 mL/min at physiologic intracranial pressures of <25 mmHg are preferred.

Generally, tissues and cells will not remain healthy if exposed to large volumes of non-physiologic ionic solutions. Accordingly, appropriate electrolyte compositions at the tissue level are important when it is considered that the circulatory method of the present invention could dilute of electrolytes from the region, to the detriment of cell membrane function. Desirably, sodium, potassium, calcium, magnesium, and chloride ions are carefully balanced in the formulations of the present invention to create, to the degree possible, normal extra-cellular compositions.

The hypothermic formulations of the invention preferably exclude four amino acids, glutathione, cysteine, ornithine and glutamine, from the group of amino acids included in the formulation, and preferably include sodium bicarbonate in an amount sufficient to increase the buffering capacity of the nutrient solution, in order to more closely resemble cerebrospinal fluid of the subject. Kits for conveniently and safely generating perfusion emulsions are described for example in U.S. patent application Ser. No. 09/619,414, filed Jul. 19, 2000 (the specific formulations and kits described therein are incorporated by reference as outlined below).

While not wishing to be limited to theory, it is believed that the albumin component help flush or perfuse from cerebrospinal tissue metabolic by products and cellular debris that accentuates the cell damage. For example, it is believed that these components help flush cytokines and TNF-α, which are believed to lead to shock.

Hypothermia

In one embodiment, for use in humans, where the CSPF is preferred, at least the following applies:

1. The CSPF contains at least a preferred amount albumin;

2. The flow rate is 20-80 mL/min;

3. The temperature of the instilled CSPF is 4-33° C. (for humans; or (i) 4° C. as a lower temperature to (ii) 4° C. below normal temperature for other animals);

4. The perfusion with CSPF is conducted over the course of 5 days or less as an adjunct to other therapy.

Another embodiment, where the cerebrospinal perfusion fluid is more preferred, at least the following applies:

1. The CSPF contains a more preferred amount of albumin;

2. The flow rate is 20-60 mL/min;

3. The temperature of the instilled CSPF is 18-33° C. (for humans; or (i) 18° C. as a lower temperature to (ii) 4° C. below normal temperature for other animals);

4. The perfusion with CSPF is conducted over 12 hours to 4 days.

Another embodiment, where the cerebrospinal perfusion fluid is specifically preferred, at least the following applies:

1. The CSPF contains 1.87 g/dL of albumin;

2. The flow rate is 20-30 mL/min;

3. The temperature of the instilled CSPF is 20-30° C. (for humans; or (i) 20° C. as a lower temperature to (ii) 7° C. below normal temperature for other animals);

4. The perfusion with CSPF is conducted over 1-2 days.

While the above listed preferences are nested in groups, it should be recognized that each of the preferences can be separately applied.

Diseases and Conditions

Neurological tissue is placed at risk of death or damage due to swelling under a variety of conditions such as stroke, ischemia, hemorrhage, or trauma. In the case of stroke, traumatic brain injury (TBI), brain hemorrhage (other than stroke), spinal cord traumatic injury, infection, ischemia caused by surgical intervention or cardiac arrest, encephalitis or encephalomyelitis not due to infection, or central nervous system ischemic injury it would be useful to protect neural tissue from death or damage. This could be done by therapeutically cooling (e.g., 18-33° C., for humans) the CNS tissue by treating the tissue with an effective amount of perfusion fluid there by inducing hypothermia in the tissue protecting it from death or damage.

In the case of infection, the treatment targets can include bacterial meningitis, including meningitis caused by Neisseria meningitides, Haemophilus influenzae type b, Streptococcus pneumoniae, E. coli, Klebsiella-Enterobacer, Listeria and group B streptococcus. Other targets are fungal meningitis and viral meningitis. Antimicrobial agents can be included in the temperature-reduced CSPF. Preferred such agents are incorporated according to the following (including preferred concentration in the CSPF):

| | | Concentration |
|---|---|---|
| Disease | Drug | (μg/mL) |
| bacterial infections | Amikacin | 1-10 |
| | Gentamicin | 1-10 |
| | Tobramycin | 1-10 |
| | Vancomycin | 3-30 |
| | Rifampin | 0.5-5 |
| | Rifamycin | 1-10 |
| | Chloramphenicol | 6-60 |
| | Amoxicillin | 1-100 |
| | Amoxicillin/clavulanate | 1-100 |
| | (Augmentin, SmithKline Beecham) | |
| | Ampicillin | 3-30 |
| | Ampicillin/sulbactam | 1-100 |
| | (Unasyn, Pfizer) | |
| | Penicillin G | 6-60 |
| | Oxacillin | 1-10 |
| | Nafcillin | 1-10 |
| | Methicillin | 5-50 |
| | Piperacillin | 6-60 |
| | Piperacillin/tazobactam | 1-100 |
| | (Zosyn, Lederle Laboratories) | |
| | Dicloxacillin | 5-50 |
| | Cefotaxime | 30-300 |
| | Cefuroxime | 30-300 |
| | Ceftriaxone | 30-300 |
| | Cefaperazone | 30-300 |
| | Ceftazidime | 30-300 |
| | Ciprofoxcin | 30-300 |
| | Erthromycin | 10-100 |
| | Streptomycin | 1-10 |
| | Isoniazid | 2-200 |
| | Ethambutol | 5-500 |
| | Ethionizmide or Ethionamide | 2-200 |
| | Pyrazinamide | 2-200 |
| | Metronidazole | 10-100 |
| | Co-Trimoxazole | 10-100 |
| Antivirals | Acyclovir | 1-100 |
| | Idoxuridine | 1-100 |
| | Nevirapine | 1-100 |
| | Didanosine | 1-100 |
| | Abacavir | 0.1-10 |
| | Zidovudine | 0.1-10 |
| | Lamivudine | 0.1-10 |
| | Indinavir | 0.1-10 |
| | Efavirenz (Sustiva ™) | 0.1-10 |
| | Ritonavir | 0.05-0.5 |
| Antifungals | Amphotericin B | 100-500 |
| | Clotrimazole | 1-100 |
| | Flucaonazole | 1-100 |
| | Itraconazole | 1-100 |
| | Ketoconazole | 1-100 |
| | Grieseofulvin | 5-500 |
| | Nystatin | 1-100 |
| | Terbinatine | 1-100 |
| | Flucytosine | 0.1-10 |
| Antimicrobials | Chloramphenicol | 10-100 |
| | Tetracycline | 1-100 |
| | Sulfadiazein | 0.5-5 |
| | Pyrimethamine | 1-100 |
| | Praziquantel | 1-100 |
| | Thiabendazole | 1-100 |

Methodology

In accordance with a preferred method of the present invention, the cool (e.g., 18-33° C., for humans) perfusion formulation is circulated through this cerebrospinal fluid route by injecting it into brain ventricles at a rate great than or equal to 20 mL/min and withdrawing it from the cisterna magna, the spinal subarachnoid space or allowed to flow from any surgical opening in the cerebrospinal pathway to nourish and to treat central nervous tissues. In other instances the fluid can be injected into the cisterna magna and withdrawn from the spinal subarachnoid space or injected into the subarachnoid space and withdrawn from another subarachnoid position.

The CSPF cooling can be introduced into the subarachnoid spaces through a catheter that transverses the skull or spinal column and the meninges. The delivery point can be the lateral ventricles, subarachnoid space around the brain, cisterna magna or anywhere along the spine. The CSPF cooling can be withdrawn from the subarachnoid space from any of these locations using a similar catheter. The CSPF cooling can be returned to the delivery system, reconditioned as necessary to add components that have been consumed or remove undesirable components that have accumulated, and then returned to the subarachnoid space in recirculating fashion. This process can be continued for days if necessary, thereby directly exposing the neuraxis to the cooling agent over an extended period of time. Where the instillation of the CSPF is via an intracranial catheter, and the exit is via a lumbar catheter, the perfusion can be termed “cranial-spinal perfusion.”

Where one seeks to flush out toxic metabolic by-products or particles, the CSPF cooling is preferably not recirculated. For example, in some embodiments, the withdrawn fluid for a first 3-5 CSF volumes is preferably not recirculated by injection at the first catheter.

This method has several advantages over global hypothermia or intravascular blood cooling. This method specifically cools the brain and/or spinal cord without significant cooling of the heart, liver, lungs, kidneys or the other parts of the body. This allows selective cooling of the brain and/or spinal to temperatures below 30° C. which avoids the risk of heart failure or blood clotting and immune system disorders. The method also provides controlled, either rapid or slow, cooling and warming of the brain and/or spinal cord.

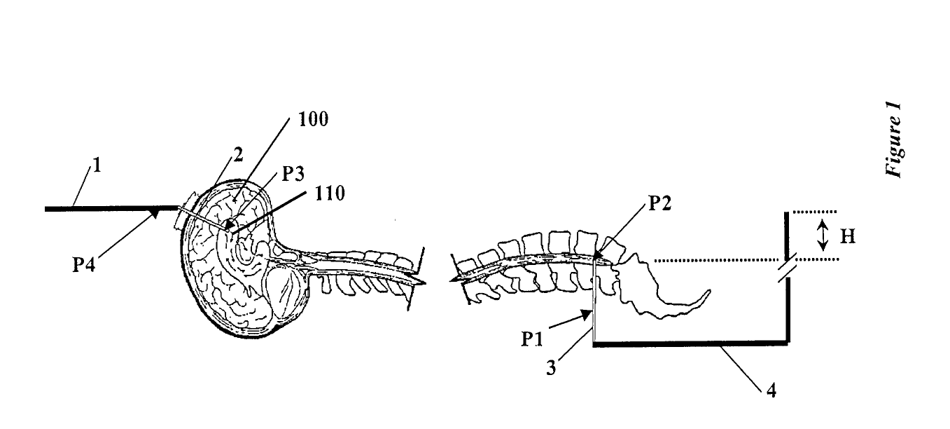

As illustrated in FIG. 1, a ventricular catheter 1 is inserted into a lateral ventrical 2. Via aqueduct 3, cisterna magna 4 and subarachnoid spaces 5, a flow pathway can be established to a lumbar outflow catheter 6. When the inflow and outflow catheters are established (typically with suitable controls to monitor intracranial and intraspinal pressure), vehicle can be used to establish the existence of a flow pathway (such as that illustrated) from the inflow catheter to the outflow catheter. The vehicle is infused under gravity feed, with the pressure head designed to avoid excessive intracranial pressure or via a perfusion device.

It will be apparent that more than two catheters can be used, though additional catheters are not particularly preferred. Care is taken to monitor the intracranial pressure to assure that flow rates do not cause excessive pressure.

Fluid for cerebrospinal perfusion is preferably perfused through the cerebrospinal pathway for a period of time to cool the brain to a therapeutic temperature, for humans, preferably between 18 and 33° C. The volume and temperature are adapted to effectively reduce the concentration of toxic by-products, or other molecular or debris components resulting from neurological damage and control tissue swelling. The volume perfused can be for example about 15 CSF volumes, where a “CSF volume” is the average volume of CSF fluid found in animals of comparable age to the subject. Preferably, at least about 2, 4, 8, 15 or 30 CSF volumes are used. In adult humans, for example, a flow rate in the range of 1,200-4,800 mL/hr is expected, resulting in the exchange of about 10-22 CSF volumes/hr. In human adults, the perfusion is preferably with 1,200 to 4,800 mL.

The perfusion can be conducted, for example, for 12, 24 or 48 or more hours. Preferably the perfusion is conducted for between 5 days or between 12 hours and 4 days. More preferably, the perfusion is conducted for at least about 48 hours. In one embodiment, the perfusion is conducted for no more than about 48 hours.

In one embodiment, the treatment is terminated in a conservative, step-wise manner, with intracranial pressure monitored. Given the significant heat input from blood (˜30% of blood flow is to the brain), the temperature established in the brain is a dynamic of the calories from blood and on-site metabolism, and heat dissipation in the cooled perfusate. Thus, temperature can be elevated by increasing the temperature of the instilled liquid, or by slowing the infusion of such heat-dissipating fluid. If the intracranial pressure sufficiently increases, the temperature elevation protocol is delayed or partially reversed. Of course, where the temperature is increased by decreasing flow, a decrease in intracranial pressure is anticipated; however, increases over the value anticipated based on prior experience or cadaver studies, the cautionary slow-down in the temperature elevating protocol is instigated.

Preferred treatment subjects among animals are mammals, preferably humans.

High Flow Rate Methods

In a preferred aspect of the invention, high flow rates, such as 20 mL/min, 25 mL/min, 30 mL/min, 40 mL/min, 50 mL/min or higher are used to assure removal of products from subarachnoid hemorrhage. Methods of achieving high flow rates are described in Barnitz, U.S. patent application Ser. No. 60/286,063, filed Apr. 24, 2001.

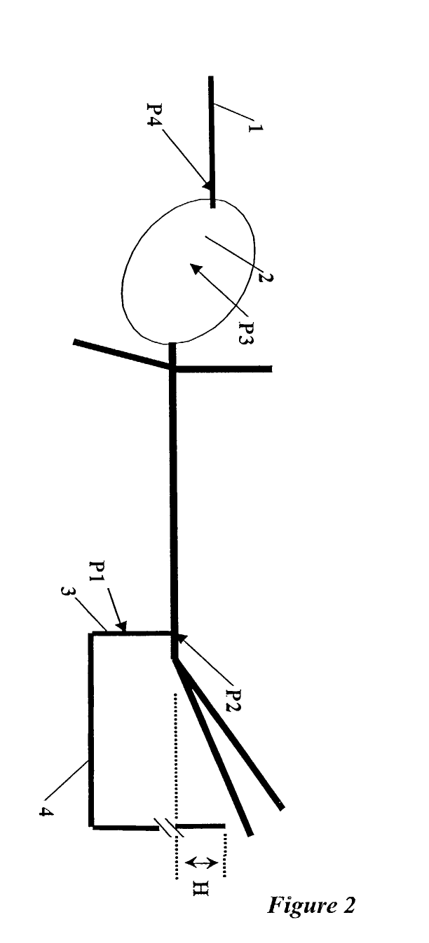

As illustrated in FIG. 2, treatment of a patient begins with the patient in supine position. Tubing 1 delivers physiologically acceptable liquid (which can be solution, suspension or emulsion) to a ventricular catheter 2, positioned in the lateral ventricle of the brain. Via the aqueduct, cisterna magna and subarachnoid spaces, a flow pathway is established to a lumbar outflow catheter 3, positioned for example at an intrathecal space of the lumbar (L4-L5) region of the spine. Any liquid that is physiologically acceptable for the central nervous system (CNS) can be used.

Pressure is monitored at the inlet to the cerebral spinal pathway, P4 (perfusion pressure, pressure at entrance to ventricular catheter), in an intracranial cavity, P3 (intracranial pressure, ICP), and at the outlet, P1 (drainage pressure, pressure at the exit of the lumbar catheter). Pressure in the spinal cord can be measured at P2 (lumbar theca pressure), or that pressure can be inferred from other data and models based on past experience. All pressures are gage values. The outlet tubing 4 can have a spill-over set at a height H (column height) relative to a zero value that is aligned with the approximate center of the spinal column. H is illustrated as at a positive value, but negative values are used after flow rates have been ramped up.

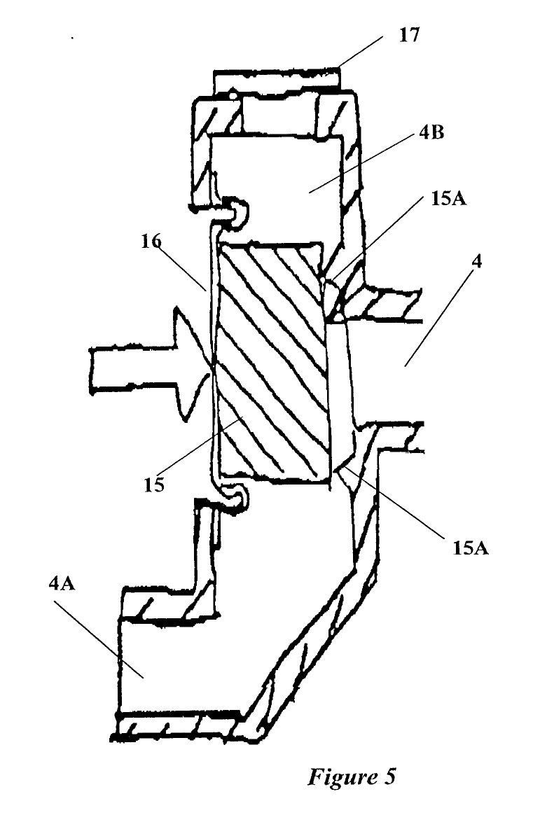

Height H is an illustrative way of setting the outlet pressure P1. Other methods include for example using pressure break devices, actively controlling the input and output pump rates, and maintaining an expansion chamber (bellows) in the outlet tubing for which the expansion, and hence the pressure, can be actively managed. One illustrative pressure break device is illustrated in FIG. 5. Outlet tubing 4 is blocked, when the break pressure has not been obtained, by barrier piece 15, which seats on rim 15A. Rolled diaphragm 16 maintains liquid isolation. The break pressure is applied on the axis indicated by the arrow, and can be set by any of a number of mechanisms known in the art, such as spring-loaded tensioning devices, electro-mechanical pushing devices, hydraulic systems pressured by pumps or electro-mechanical pushing devices, gas pressure, and the like. In the illustrated break device, sterile filter 17 allows for gas (e.g., air) intake to manifold 4B, which is connected (independent of barrier piece 15) to outlet tubing 4A. To allow for negative pressure, the pressure break device can be positioned sufficiently below the H=0 level so that easing the break pressure effectively brings PI to an appropriate negative value. Or, sufficient pumping can be applied to the fluid in outlet tubing 4A (in the absence of a gas intake) to maintain the desired negative pressure. Pressure control can be through active relative control of pumps (e.g., using the feedback loops and controller discussed below) or manual.

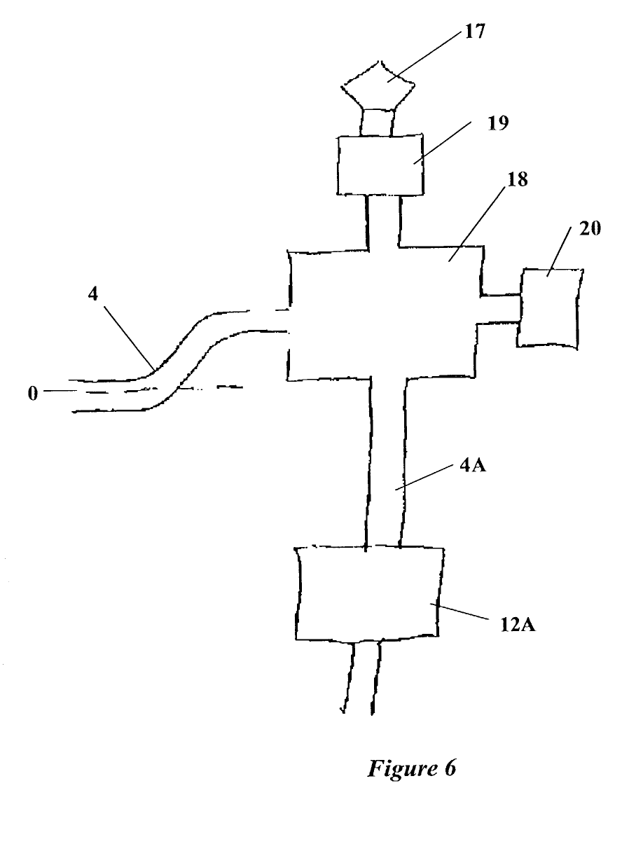

Another illustration of a pressure control device is found in FIG. 6. Manifold 18 is rigid, and can thus maintain a partial reduced pressure (measured against atmospheric). Manifold 18 is preferably placed above (e.g., 5 cm, measured from the bottom of the manifolds connection to tubing 4) the H=0 level. Valve 19 (if present) controls any introduction of gas into manifold 18, and can be for example a variable resistance valve or an adjustable pressure relief valve. Pressure monitor 20 can be a pressure transducer. Recycle pump 12A is suitable to create a reduced pressure in manifold 18. Preferably, pressure control is by active feedback control of recycle pump 12A, based on pressure data, for example from pressure monitor 20.

After initial setup of the catheters, the above introduced parameters can be, with no flow, for example:

| Body Position | H | P1 | P2 | P3 | P4 |
|---|---|---|---|---|---|
| Horizontal | +5 cm | 4 mmHg | 4 mmHg | 4 mmHg | 4 mmHg |

The values after initiation of flow are as set forth below for various flow rates. These values are based on the use of a 14 gauge catheter as the lumbar catheter. Exemplary catheters are described, for example, in co-pending Ser. No. 09/382,136, filed Nov. 26, 1999. The flow resistance of this catheter is a major determinant of P2, and consequently of P3. The use of catheters of different flow resistances will modify the pressure relationships as can be determined with appropriate calculations and modeling. When flow is at 10 mL/min:

| Body | H | P1 | P2 | P3 | P4 |
|---|---|---|---|---|---|
| Position | (cm) | (mmHg) | (mmHg) | (mmHg) | (mmHg) |
| Horizontal | +5 | 4 mmHg | 12 mmHg | 16.5 mmHg | 19.0 mmHg |
| Horizontal | 0 | 0 mmHg | 8 mmHg | 12.5 mmHg | 15.0 mmHg |
| Horizontal | −5 | −4 mmHg | 4 mmHg | 8.5 mmHg | 11.0 mmHg |

When flow is at 20 mL/min:

| Body | H | P1 | P2 | P3 | P4 |
|---|---|---|---|---|---|
| Position | (cm) | (mmHg) | (mmHg) | (mmHg) | (mmHg) |
| Horizontal | 0 | 0 | 16.75 | 27.0 | 32.25 |
| Horizontal | −10 | −8.0 | 8.75 | 19.0 | 24.25 |
| Horizontal | −15 | −12.0 | 4.75 | 15.0 | 20.25 |
| Horizontal | −20 | −16.0 | 0.75 | 11.0 | 16.25 |

When flow is at 30 mL/min:

| Body | H | P1 | P2 | P3 | P4 |
|---|---|---|---|---|---|
| Position | (cm) | (mmHg) | (mmHg) | (mmHg) | (mmHg) |
| Horizontal | −10 | −8.0 | 19.75 | 35.75 | 45.25 |
| Horizontal | −20 | −16.0 | 11.75 | 27.25 | 37.25 |
| Horizontal | −30 | −24.0 | 3.75 | 19.75 | 29.25 |

The shaded values are to be avoided. P2 values of less than about 3.5 are typically avoided.

The central nervous system (CNS) physiologically acceptable liquid used in the above example is a fluorocarbon nutrient emulsion containing eight a constituent compositions is as set forth in the table below for a 1200 mL unit of the emulsion. However, as mentioned, any CNS physiologically acceptable fluid can be used with this invention.

| | Amount |
|---|---|
| Constituent Compositions | g/unit |
| t-Bis-perfluorobutyl ethylene | 200 |
| NaCl, USP | 7.63 |
| NaHCO₃, USP | 2.19 |
| Purified egg yolk phospholipid, | 13.8 |
| KCl, USP | 0.23 |
| MgCl₂-6H₂O, USP | 0.24 |
| CaCl₂-2H₂O, USP | 0.18 |
| Dextrose, USP | 1 |
| Albumin (Human), USP | 20 |
| L-lysine HCl, USP | 0.0032 |
| L-alanine, USP | 0.0034 |
| L-serine, USP | 0.0030 |
| L-threonine, USP | 0.0036 |
| L-arginine, USP | 0.0022 |
| L-leucine, USP | 0.0015 |
| L-isoleucine, USP | 0.0006 |
| L-valine, USP | 0.0020 |
| L-phenylalanine, USP | 0.0010 |
| L-tyrosine, USP | 0.0010 |
| L-histidine, USP | 0.0012 |
| L-methionine, USP | 0.0003 |
| NaH₂PO₄, USP | 4.1 |
| Na₂HPO₄, USP | 0.61 |
| α-ketoglutaric acid | 0.030 |
| Sterile Water for Injection, USP | 1040 mL |

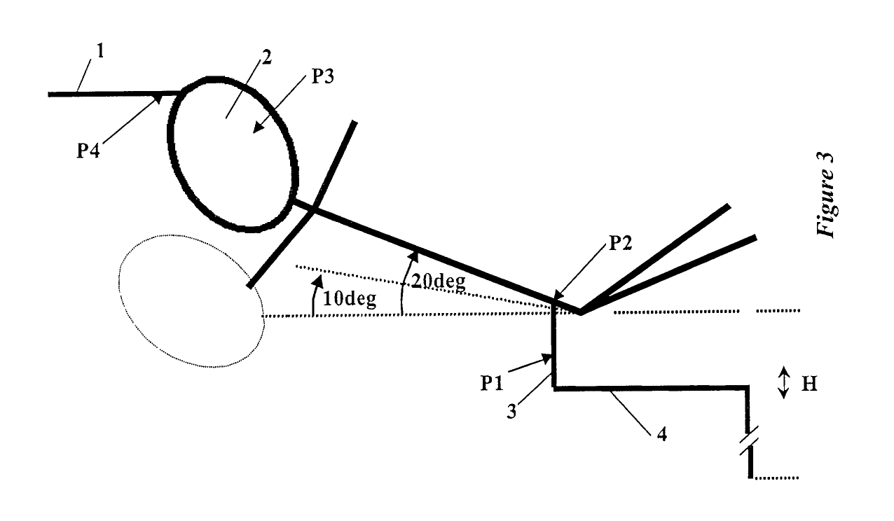

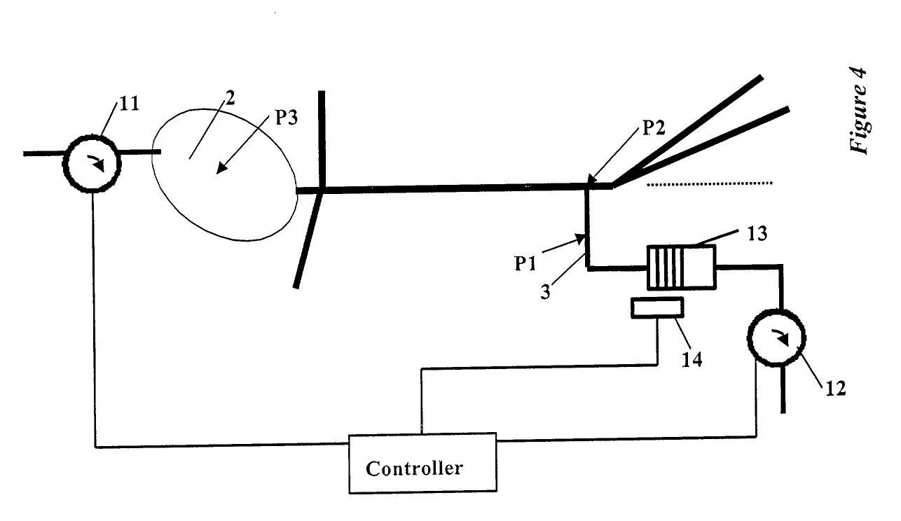

FIG. 3 shows elements of FIG. 2 in a more schematic fashion. After higher flow has been initiated, the patient can be elevated as indicated in FIG. 4. For example, the patient can be safely inclined when flow rates have become high, such as 20 mL/min, 25 mL/min, 30 mL/min or higher. In FIG. 4, the patient is illustrated at a 20 degree incline, with a 10 degree incline illustrated in dotted lines. Incline angles are often in the lower range of, for example, 10 or 20 degrees, but higher inclinations can be used to achieve still more elevated flow rates, such as 50, 60 or 70 mL/min.

Exemplary pressure parameters with an incline are illustrated below. Body position only affects ICP (P3) and perfusion pressure (P4), lumbar theca(P2) and drainage(P1)pressures are unaffected. A five degree incline will reduce ICP and PP by 3.75 mmHg for an average sized patient, by 7.25 mmHg for a 10 degree incline, by 11.0 mmHg for a fifteen degree incline, and by 14.50 mmHg for a twenty degree incline. For example, when flow is at 30 mL/min:

| Body | H | P1 | P2 | P3 | P4 |
|---|---|---|---|---|---|
| Position | (cm) | (mmHg) | (mmHg) | (mmHg) | (mmHg) |
| Horizontal | −30 | −24.0 | 3.75 | 19.8 | 29.3 |
| 5° incline | −30 | −24.0 | 3.75 | 16.0 | 25.5 |
| 10° incline | −30 | −24.0 | 3.75 | 12.5 | 22.0 |

In another aspect, the delivery algorithm takes into account a phenomenon (and risk) involved in recycling the liquid that has cycled through the cerebral spinal pathway back through the pathway. A mismatch in inflow and outflow rates can occur, resulting from the tolerances in the two pumping systems, a difference in CSF production and absorption, or a change in ICP and the concurrent change in CNS volume due to compliance in the CNS. Such a mismatch could lead to an over or under pressure condition in the patient. This risk is addressed in one or more of two ways.

First, the flow rate of pumping of the effluent is maintained a rate sufficiently higher than the delivery flow rate to account for these sources of variation. A gas/air intake (preferably fitted with a sterile filter) in the effluent line provides a fluid source to account for the higher flow rate. The intake is linked to the tubing/plumbing before the pump inlet. Before recycle, the liquid can be passed through a holding container in which the extra gas is separated away (preferably through a sterile filter). This format is effective to not, of itself, create a significant negative pressure. The minor pressure differential across the sterile filter is not a significant pressure. A device for accomplishing these functions with peristaltic pumps is described in copending U.S. application Ser. No. 60/286,057, filed Apr. 24, 2001. A preferred set-point in the flow differential is between 5 and 15%, such as about 10%. Note, that this is the differential set with respect to the average calibrated flow rate, but in some instances the differential is established in part in acknowledgement that the pumps used for reliable, non-pulsatile, sterile pumping can be somewhat variable in their actual flow rate.

Second, as illustrated in FIG. 5, a bellows 13 is incorporated into the tubing/plumbing before the recycle pump 12, and the expansion or contraction of the bellows is monitored by monitor 14. Monitor 14 sends data to the controller, which adjusts the rate of delivery pump 11 or recycle pump 12 as appropriate. Data from pressure monitoring devices can also be sent to the controller, so that the controller can avoid increasing the flow of delivery pump 11, or reduce the flow of delivery pump 11, in response to pressure data.

The monitor 14 can be physically connected to the bellows via a linear transducer or linear potentiometer, providing an electrical signal for the amount of movement in the bellows. Or, the monitor can monitor the offset of the below with a light reflectance angle, with multiple reflectance monitors that indicate whether the bellows is within or without a reflectance pathway, by measuring the distance analog of an acoustic reflectance. Other methods recognized in the art for measuring displacements can be used. Where a bellows such as illustrated functions to control pressure at the drainage end, the same devices for controllably applying pressure as discussed above with reference to the break pressure can be used to exert the required force on the bellows.

Definitions

The following terms shall have, for the purposes of this application, the respective meanings set forth below.

effective amount: The meaning of “effective amount” will be recognized by clinicians but includes an amount effective to reduce, ameliorate or eliminate one or more symptoms of the disease sought to be treated or the condition sought to be avoided or treated, or to otherwise produce a clinically recognizable change in the pathology of the disease or condition.

normal temperature. A normal temperature of a mammal is the midpoint of the recognized normal temperature range as measured orally. For humans, the normal temperature is 37° C.

nutrient-providing effective amount. A nutrient-providing effective amount of a substance is an amount that can be expected, provided sufficient amounts of other nutrients, to increase metabolism or reproduction of mammalian cells compared with nutrient solutions lacking that substance.

oncotic agent. By oncotic agent is meant substances, generally macromolecules, that are of a size that is not readily able to leave the body cavity or other fluid containing body spaces (such as the cerebrospinal pathway, including the cerebral ventricles and subarachnoid spaces) into which they are inserted. Such oncotic agents are exemplified by blood plasma expanders which are known in general as macromolecules having a size sufficient to inhibit their escape from the blood plasma through the circulatory capillary bed into the interstitial spaces of the body. Serum albumin, preferably human serum albumin, is one well known blood plasma protein that can be used as an oncotic agent. Polysaccharide blood plasma expanders are often glucan polymers. For example, Hetastarch (a product of American Home Products) is an artificial colloid derived from a waxy starch composed almost entirely of amylopectin with hydroxyethyl ether groups introduced into the alpha (1-4) linked glucose units. The colloid properties of a 6% solution (wt/wt) of hetastarch approximate that of human serum albumin. Other polysaccharide derivatives may be suitable as oncotic agents in the blood substitute according to the invention. Among such other polysaccharide derivatives are hydroxymethyl alpha (1-4) or (1-6) polymers and cyclodextrins. In general, it is preferred that the polysaccharide is one that is non-antigenic. High molecular weight agents such as Dextran 70 having a molecular weight of about 70,000 Daltons are generally less preferred because they increase viscosity of the colloidal solution and impair the achievement of high flow rates. Preferably, the oncotic agent is in an amount effective to provide, in conjunction with other components of a fluorocarbon nutrient emulsion or a nutrient solution, an oncotic pressure of one to seven torr.

respiration. Respiration is the physical and chemical processes by which an organism supplies its cells and tissues with the oxygen needed for metabolism and, preferably, relieves them of the carbon dioxide formed in energy-producing reactions.

respiration-supporting amount. A respiration-supporting amount of oxygen is an amount that would, in model experiments, provide a statistically significant reduction in morbidity following a focal ischemic event.

Where ranges are given as appropriate or preferred, and narrower ranges are also provided, it should be recognized that any upper limit can be paired with any lower limit (or vice versa) to define another preferred range. Moreover, if in presenting focused recitations of preferences for one factor in combination with preferences for another factor, it will be recognized that, unless there is a particular contraindication, all combinations of preferences are themselves preferred.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the maimer described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of treating, in an animal, an injury that is stroke, traumatic brain injury including traumatic surgical brain injury, brain hemorrhage other than stroke, spinal cord traumatic injury including spinal cord surgical trauma, infection, ischemia caused by surgical intervention, CNS injury caused by cardiac arrest, encephalitis or encephalomyelitis not due to infection, comprising:
creating a flow pathway, with an instillation catheter and an effluent catheter, that provides liquid flow in the vicinity of the injury;
instilling into the flow pathway an cerebrospinal perfusion fluid having a temperature from (i) 4° C. to (ii) 4° C. below normal temperature of the animal; and
maintaining perfusion through the flow pathway for at least 12 hours of the cerebrospinal perfusion fluid having a temperature, at the point of instillation, from (i) 4° C. to (ii) 4° C. below normal temperature of the animal.

2. The method of claim 1, wherein the animal is a mammal.

3. The method of claim 2, wherein the mammal is a human.

4. A method of treating a brain or spinal injury comprising:
creating a flow pathway, with an instillation catheter and an effluent catheter, that provides liquid flow in the vicinity of the injury;
instilling into the flow pathway a cerebrospinal perfusion fluid having a temperature from (i) 4° C. to (ii) 4° C. below normal temperature, wherein the cerebrospinal perfusion fluid does not have a respiration-supporting amount of oxygen; and
maintaining perfusion of the cerebrospinal perfusion fluid having a temperature from (i) 4° C. to (ii) 4° C. below normal temperature through the flow pathway for at least 12 hours.

5. The method of claim 4, wherein the liquid flow of 20 to 60 mL/min.

6. The method of claim 5, wherein the cerebrospinal perfusion fluid has and is maintained at a temperature from (i) 18° C. to (ii) 4° C. below normal temperature.

7. The method of claim 4, wherein the liquid flow of 20 to 30 mL/min.

8. The method of claim 7, wherein the cerebrospinal perfusion fluid has and is maintained at a temperature from (i) 18° C. to (ii) 4° C. below normal temperature.

9. The method of claim 4, wherein the animal is a mammal.

10. The method of claim 9, wherein the mammal is a human.

11. The method of claim 4, further comprising:
monitoring the temperature of effluent cerebrospinal perfusion fluid from the flow pathway;
providing a signal when temperature of the effluent reaches 15° C., [or 17 or 18° C.] the signal comprising (a) a feedback signal for a controller that decreases the flow rate of instillation or increases the temperature of the instilled cerebrospinal perfusion fluid, or (b) an alarm.

12. The method of claim 4, wherein the injury is stroke, traumatic brain injury including traumatic surgical brain injury, brain hemorrhage other than stroke, spinal cord traumatic injury including spinal cord surgical trauma, infection, ischemia caused by surgical intervention, CNS injury caused by cardiac arrest, encephalitis or encephalomyelitis not due to infection.

13. The method of claim 12, wherein the animal is a mammal.

14. The method of claim 13, wherein the mammal is a human.

15. A method of treating in an animal a brain or spinal injury, comprising:
creating a flow pathway, with an instillation catheter and an effluent catheter, that provides liquid flow in the vicinity of the injury;
instilling into the flow pathway an cerebrospinal perfusion fluid having a temperature from (i) 4° C. to (ii) 4° C. below normal temperature of the animal;
thereafter gradually, for a pre-designated amount of time or pursuant to a pre-designated schedule, (i) elevating the temperature of the instilled cerebrospinal perfusion fluid or (ii) decreasing the flow rate of the instilled cerebrospinal perfusion fluid to elevate the temperature of cerebrospinal perfusion fluid exiting the effluent catheter; and
in conjunction with the gradual elevating or decreasing, monitoring intracranial pressure and, should the intracranial pressure indicate undue swelling of neural tissue, doing one or more of (i) maintaining a temperature or flow rate for more than the pre-designated time or delaying the schedule of elevating temperature or decreasing flow, (ii) decreasing the temperature of the instilled cerebrospinal perfusion fluid, or (iii) increasing the flow rate of the instilled cerebrospinal perfusion fluid.

16. A method of delivering an cerebrospinal perfusion fluid into the cerebral spinal pathway of an animal, comprising:
initiating flow of the cerebrospinal perfusion fluid into a ventricular catheter, through a cerebral spinal pathway, and out a lumbar outflow catheter with the patient in a supine position and using a first flow rate, wherein a positive first value for an outlet pressure is maintained in plumbing from the lumbar outflow catheter;
increasing flow to a second flow rate greater than the first in conjunction with decreasing the outlet pressure to a second, negative value; and
thereafter maintaining flow of the cerebrospinal perfusion fluid into the ventricular catheter with the temperature of the physiologically acceptable liquid from (i) 4° C. to (ii) 4° C. below normal temperature of the animal.

17. The method of claim 16, further comprising:
between initiating flow to the first rate and increasing to the second rate, adjusting the flow rate to an intermediate rate between the first and second rate in conjunction with adjusting the outlet pressure to a value between the first and second value.

18. The method of claim 16, further comprising:
at some point after finishing the flow at the first rate and prior to the maintaining, inclining the patient.

19. The method of claim 16, wherein the second flow rate is 20 mL/min or higher.

20. The method of claim 16, wherein the cerebrospinal perfusion fluid is recycled through the cerebral spinal pathway by pumping with a first pump liquid into the cerebral spinal pathway and pumping with a second pump efflux from the cerebral spinal pathway for use in recycling, wherein the method further comprises operating one or both of the following safety procedures:
(a)
operating the second pump at a flow rate target T2 higher than the operating target flow rate TI of the first pump, and
drawing gas into the efflux flow as needed to prevent the flow rate of the second pump from creating a significant negative pressure,
wherein the higher rate of the second pump is adapted to prevent variance in the flow rates of the pumps or in compliance of a flow path from the first to the second pump from allowing the second pump to operate as a flow constrictor; or
(b)
providing a bellows in an efflux flow pathway,
lowering the T1:T2 ratio if the bellows expand or expand beyond an alarm-generating value, and
increasing the T1:T2 ratio if the bellows contract or contract beyond an alarm-generating value.
